(19) United States
(12) Patent Application Publication
(10) Pub. No.: US 2022/0257146 A1
(43) Pub. Date: Aug. 18, 2022

(54) SYSTEM AND METHOD FOR MOTION ANALYSIS

(71) Applicant: NATIONAL UNIVERSITY OF SINGAPORE, Singapore (SG)

(72) Inventors: Ronald Boyd ANDERSON, Singapore (SG); Ye WANG, Singapore (SG)

(21) Appl. No.: 17/621,737

(22) PCT Filed: Jun. 25, 2020
(86) PCT No.: PCT/SG2020/050360

§ 371 Date: Dec. 22, 2021

(30) Foreign Application Priority Data
Jul. 5, 2019 (SG) 10201906276U

(51) Int. Cl. A61B 5/11; A61B 5/05; A61B 5/00; G01S 13/62; G01S 13/08; G01S 13/02; G01S 1/04

(52) U.S. Cl. CPC A61B 5/112; A61B 5/1126; A61B 5/05; G01S 1/0428; G01S 13/62; G01S 13/08; G01S 13/0209; A61B 5/6829

(57) ABSTRACT

A system for motion analysis of a subject includes two or more sensor units that are attachable at respective attachment points of the subject to detect motion of the attachment points relative to each other, each sensor unit including a time-of-flight (TOF) ranging sensor in communication with at least one processor. The at least one processor is configured to: cause the sensor units to execute a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and determine, from at least the TOF distance data, one or more motion metrics.

### TECHNICAL FIELD

The present invention relates, in general terms, to a system and method for motion analysis, such as gait analysis.

### BACKGROUND

In various contexts it is desirable to be able to measure movements of an individual, for example to perform biomechanical assessments to improve athletic performance, or to test for physical behaviours that may be characteristic of an underlying neurological condition.

For example, gait analysis is the measurement of quantities related to human locomotion (for example step time, or stride length). These quantities are known as spatiotemporal gait parameters. Variability of these gait parameters is an important diagnostic indicator of health, correlating with both quality of life and mortality, and is of great interest to both clinicians and researchers. As such, there exists a multitude of technologies for measuring and quantifying gait, including instrumented walking mats, treadmills, motion capture systems, and wearable sensors (such as pressure sensitive foot switches or inertial sensors). These technologies have different strengths and weaknesses.

For example, instrumented walking mats such as the GAITRite of CIR Systems, Inc. (Franklin, N.J.) provide both spatial and temporal gait analysis, at the cost of requiring a relatively large area for use. By contrast, Inertial Measurement Unit (IMU) based wearable sensors such as the Shimmer3 (shimmersensing.com) sacrifice measurement accuracy and comprehensiveness for portability and practicality.

Unlike most walking mats, treadmills, or motion capture clinical gait analysis systems, IMU-based wearable sensors allow for a more convenient and practical way to perform gait analysis outside of a laboratory or hospital setting. As such, these sensors can be used to capture the natural walking of elderly persons or those with neurological conditions. However, some measures of gait variability of clinical interest are difficult to estimate using IMU-based sensors. One reason for this limitation is because the sensors only measure acceleration and rotation, and to infer even simple gait parameters (such as stride length), complicated models must be employed. One important gait parameter that is difficult to estimate with IMU-based sensors is step width, which is an indicator of fall risk. Another is estimating the placement of the foot in terms of base-of-support during walking, which can help in evaluating balance and subsequently fall risk.

Wearable IMU-based sensors are widely used, and have been used to record the gait of healthy elderly people, and those with neurological conditions such as Parkinson's Disease. These systems can measure various gait parameters, including step time, step length, step time variability and step length variability. However, other gait parameters are harder to estimate reliably, such as swing time or step width. Additionally, sensor location, speed, and the algorithms employed in analysis have a direct effect on the accuracy of any gait parameter estimation.

It would be desirable to overcome or alleviate at least one of the above-described problems, or at least to provide a useful alternative.

### SUMMARY

Disclosed herein is a system for motion analysis of a subject, including:
two or more sensor units that are attachable at respective attachment points of the subject to detect motion of the attachment points relative to each other, each sensor unit including a time-of-flight (TOF) ranging sensor in communication with at least one processor;
wherein the at least one processor is configured to:
   cause the sensor units to execute a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
   determine, from at least the TOF distance data, one or more motion metrics.

Also disclosed herein is a method of motion analysis of a subject, including:
attaching two or more sensor units at respective attachment points of the subject to detect motion of the attachment points relative to each other, each sensor unit including a time-of-flight (TOF) ranging sensor in communication with at least one processor;
executing, by the at least one processor, a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
determining, from at least the TOF distance data, one or more motion metrics.

Further disclosed herein is at least one computer-readable medium storing machine-readable instructions that, when executed by at least one processor, cause the at least one processor to perform a method as disclosed herein.

### BRIEF DESCRIPTION OF THE DRAWINGS

Embodiments of the present invention will now be described, by way of non-limiting example, with reference to the drawings in which:

FIG. 1 is a schematic diagram of a sensor configuration in an example system for motion analysis;

FIG. 2 is a block diagram of a sensor unit of the system of FIG. 1;

FIG. 3 is an illustration of certain gait parameters measurable using embodiments of the invention;

FIG. 4 illustrates geometry of accelerometer axes of a sensor unit of the system of FIG. 1;

FIG. 5 shows plots of battery life of a sensor unit in two different scenarios;

FIG. 6 is an example architecture of a mobile computing device of the system 10 of FIG. 1;

FIG. 7 shows messaging flow in a ranging protocol implemented by a system for motion analysis;

FIG. 8 is a schematic illustration of a sequence of steps in the ranging protocol of FIG. 7;

FIG. 9 illustrates poll and response messages transmitted between a pair of sensors in a system for motion analysis;

FIG. 10 is a flow diagram of an exemplary ranging protocol;

FIG. 11 shows (a) unfiltered and (b) filtered signals measured by an exemplary system for motion analysis, plotted against measurements obtained by a prior art system;

FIG. 12 illustrates sensor geometry in a motion analysis system according to certain embodiments;

FIG. 13 illustrates ranging sensor geometry during a left step taken by a subject;

FIG. 14 shows general disposition of sensors of a motion analysis system relative to each other during a step;

FIG. 15 shows inter-sensor distance measurements as a function of time for a right step followed by a left step;

FIG. 16 is a flow diagram of an exemplary method for determining gait metrics;

FIG. 17 shows foot positions of a subject measured by a system according to embodiments of the invention, overplotted on foot positions measured by a prior art system;

FIG. 18 is an annotated example of a sensor signal, with vertical lines representing step times measured by a prior art system;

FIG. 19 shows measurement error for gait metrics obtained using an embodiment of the invention that makes use of an extended Kalman filter;

FIG. 20 shows a sensor signal obtained using an embodiment of the invention, with a Savitsky-Golay filtered signal overplotted on the raw sensor signal;

FIG. 21 shows a sensor signal obtained using an embodiment of the invention, with an EKF-filtered signal overplotted on the raw sensor signal;

FIG. 22 shows kinematics of sensor movement used for generating an EKF model;

FIG. 23 shows measurement error for gait metrics obtained using an embodiment of the invention that makes use of support vector regression; and

FIG. 24 shows measurement error for gait metrics obtained using an embodiment of the invention that makes use of a multilayer perceptron.

### DETAILED DESCRIPTION

Embodiments of the invention generally relate to the use of multiple, wearable time-of-flight (TOF) ranging sensors to measure distances, at a succession of times, between parts of a subject that are in motion relative to each other. For example, the sensors may be attached to the feet of the subject to conduct gait analysis.

The TOF ranging sensors may be RF-based sensors, such as Ultra Wideband (UWB) radio sensors. Alternatively, they may be laser ranging, infrared or ultrasonic sensors.

Measurements recorded by the TOF sensors can be used to determine motion metrics, such as gait parameters including stride width and foot placement, which are difficult or even impossible to measure accurately with inertial measurement unit (IMU) sensors. In some embodiments, measurements from the TOF sensors may be combined with measurements from inertial sensors to improve the accuracy of the motion metric determination.

As used herein, a “motion metric” means one or more numerical values indicative of the motion of a subject over one or more time segments (which may be of variable duration). For example, a motion metric may be a gait metric such as step time, step length, step width, stride time, stride length, stride velocity, cadence, swing time, or swing length; or another parameter, such as arm swing, elbow extension, neck rotation, knee extension, or postural sway. A motion metric may also be a summary value characteristic of the one or more numerical values, such as a mean or standard deviation (or other measure of location or variability).

In certain embodiments, each TOF sensor measures the distances to two sensors on the other foot, allowing an accurate assessment of foot placement relative to the other foot. Unlike systems such as GAITRite, this placement can be calculated throughout the stride. Advantageously, embodiments of the present invention combine the spatial accuracy benefits of instrumented walking mats with the portability benefits of IMU-based wearable sensors.

Embodiments of the invention provide one or more of the following benefits:
estimation of clinically significant gait parameters that are not currently determinable by IMU-based wearables, such as step width and spatial foot placement;
accurate estimation of gait metrics using methods of low computational complexity; and
improved estimates of gait metrics via the combination of data from IMU and TOF sensors.

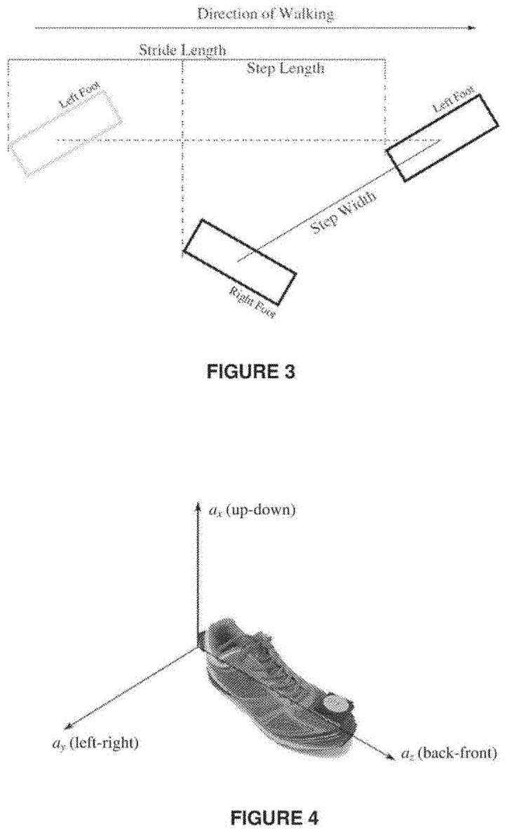

In the following discussion, and for ease of direct comparison with existing systems such as the GAITRite walking mat, the following definitions for some important gait metrics are adopted (see FIG. 3).
When one foot is off the ground this is known as single support time, and when both are on the ground this is known as double support time.
A heel strike is defined as the time the heel of the foot makes contact with the ground. Likewise, a toe off is defined as the time the toe of the foot leaves contact with the ground.
Step Time is the duration between two heel strikes of alternating feet.
Step Length is the distance (in direction of movement) between two consecutive placements of alternating feet.
Step Width is the diagonal distance between the mid-point of the feet during double support time.
Stride Time is the duration between two consecutive heel strikes of the same foot.
Stride Length is the distance between two consecutive placements of the same foot.
Stride Velocity is the ratio of stride length to stride time.
Cadence is the number of steps made in a fixed time period (commonly measured in steps per minute).
Swing Time is the duration spent during the swing phase (when the foot is not in contact with the ground).
Stance Time is the duration spent during the stance phase (when the foot is in contact with the ground).

These metrics, and their variability, are all useful to clinicians for diagnosis of neurological conditions or in evaluating general health.

### System for Gait Analysis 10

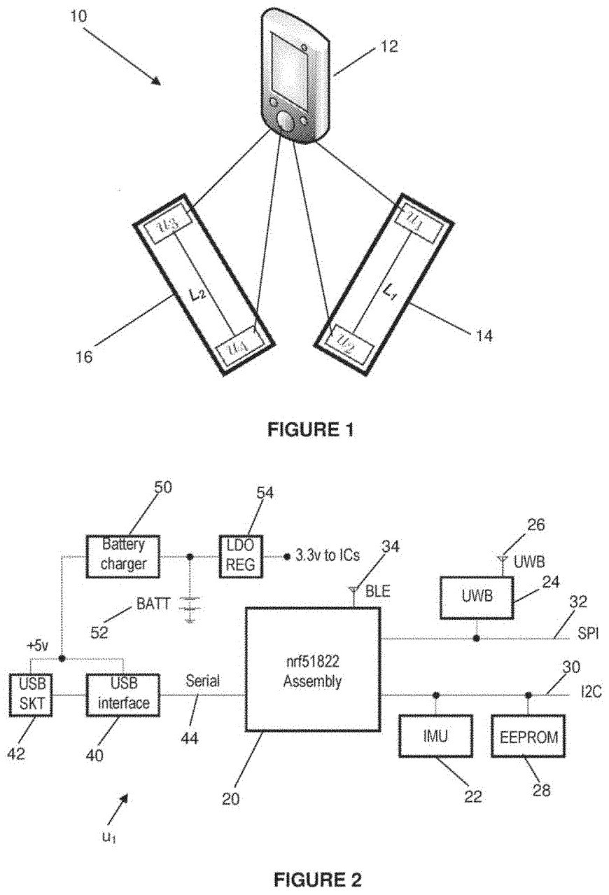

In certain embodiments, with reference to FIG. 1, a system 10 includes two or more sensor units. The sensor units in FIG. 1 are designated as u₁ to u₄, though it will be appreciated that there may be more than four sensor units, or as few as two or three.

The sensor units u₁ to u₄ are attachable at respective attachment points of a subject. In the particular example shown in FIG. 1, a first pair of sensor units u₁ and u₂ is attached to a first foot 14 of the subject, and a second pair of sensor units u₃ and u₄ is attached to a second foot 16 of the subject. It will be appreciated that just a single sensor unit (for example, u₁) could be attached to the first foot 14, or another body part, and another, single, sensor unit (for example, u₃) could be attached to the second foot 16, or another body part. What is important is that at least two of the sensor units should be placed at different points of the subject that will be in motion relative to each other, so that such sensor units can be used to measure the relative motion between the attachment points, and thereby provide information that can be used to infer at least one motion metric.

In the particular example shown in FIG. 1, the first and second sensor units u₁, u₂ are in spaced arrangement at a first spacing L₁ on a first foot 14 of the subject. For example, each sensor unit u₁, u₂ may include a housing having means (such as a clip, band, hook-and-loop fastener, etc.) for attaching the sensor unit to the subject (e.g., to an article of clothing, a belt, a shoe, etc.). Alternatively, the sensor units u₁, u₂ may each be embedded in an article of clothing or shoe worn by the subject. The first sensor unit u₁ may be placed at or near a toe of the shoe and the second sensor unit u₂ at or near a heel of the shoe, for example in the sole of the shoe or on an outer surface of the shoe. In some embodiments the sensors may comprise components that are woven into an article of clothing or a shoe.

In the embodiment illustrated in FIG. 1, the system 10 also includes a third sensor unit u₃ and a fourth sensor unit u₄ for placement in spaced arrangement at a second spacing L₂ on a second foot 16 of the subject. Typically, the second spacing L₂ will be the same as the first spacing L₁, i.e. L₁=L₂=L. As for the first and second sensor units, the third and fourth sensor units u₃, u₄ may include housings that are attachable to a shoe, or embedded in the shoe.

Each of the first u₁, second u₂, third u₃ and fourth u₄ sensor units includes a time-of-flight (TOF) ranging sensor, such as a UWB ranging sensor. Each TOF ranging sensor is in communication with at least one processor. For example, each sensor unit may include at least one on-board processor that communicates with its TOF ranging sensor via a bus.

The at least one processor is configured to cause the sensor units u₁, u₂, u₃, u₄ to execute a two-way ranging protocol at a succession of times. The two-way ranging protocol includes transmission of one or more signals from, and reception of one or more signals at, the TOF ranging sensors, to determine TOF distance data indicative of respective distances between the sensor units at respective times. An exemplary two-way ranging protocol is described in more detail below.

The at least one processor is also configured to determine, from at least the TOF distance data, one or more motion metrics, such as one or more gait metrics.

The system 10 may include at least one processor external to the sensor units, such as a processor of an external computing device (e.g. a mobile computing device 12) with which the sensor units communicate, e.g. via Bluetooth or another wireless communications protocol. As such, operations performed by the components of the sensor units, including computation of distances and motion metrics, may be carried out or instructed by on-board processors of the sensor units themselves, and/or by processors of external computing devices with which the sensor units communicate. For example, the sensor units may collectively execute the ranging protocol, compute the relative distances between the sensor units, and store the computed distances in on-board memory for later transmission to an external computing device, a processor of which may then use the computed distances to determine the one or more motion metrics.

### Sensor Unit u₁

An example sensor unit u₁ is shown in FIG. 2. It will be appreciated that the other sensor units u₂, u₃, u₄ may be substantially identical in construction to sensor unit u₁.

Sensor unit u₁ may include an on-board processor 20 that is in communication with a memory that stores computer-readable instructions executable by the on-board processor, and that may also store data recorded by one or more sensors of the sensor unit u₁. In one example, the processor is part of a system-on-a-chip (SoC) such as a core nrf51822-based assembly of Nordic Semiconductor, with a 32-bit ARM Cortex M0 at 16 MHz, with 24 kB of RAM, and 128 Kb of flash memory. An SoC, in the context of a sensor unit, may be referred to as a “processor” in the discussion below.

In addition, sensor unit u₁ includes an inertial measurement unit (IMU) 22, such as a 6-axis IMU MPU6050 providing three channels from an accelerometer (aₓ, a_{y}, a_{z}) and 3 channels from a gyroscope (gₓ, g_{y}, g_{z}). The IMU 22 and an EEPROM 24 are in communication with processor 20 via an I²C bus 30.

The sensor unit u₁ also has a TOF ranging sensor, for example a UWB sensor 24 having an antenna 26 and being in communication with processor 20 via a serial peripheral interface (SPI) bus 32. The UWB sensor 24 may be a real-time location module such as the DWM1000 of Decawave Limited. UWB sensor 24 can be used for absolute range measurements. The ranging sensor 24 may be configured in a low-power mode, with a transmission rate of 6800 Kbps, and a pulse repetition frequency of 64 MHz, for example.

Sensor unit u₁ may communicate with external computing devices, such as mobile device 12, via one or more interfaces such as a Bluetooth 4.2 (BLE) interface 34 and a USB interface 40 (that is connected to processor 20 via serial bus 44). The USB interface 40 may also be used to charge a rechargeable battery (such as a 110 mAh LiPo battery) 52. To this end, sensor unit u₁ includes battery management circuitry (including charger 50 and low-dropout regulator 54) to support ultra-low power use cases, and for measuring and charging the battery 52.

In certain embodiments, the sensor unit u₁ may have its electronic components mounted to a single-sided 20.4 mm×24.1 mm PCB, which as mentioned above, may be housed in a case for mounting on a shoe or another suitable attachment point of the subject (e.g. a belt, article of clothing, etc.).

The sensors u₁, u₂, u₃, u₄ can be used to measure distances as well as motion. The configuration of the sensors and their locations on the body may determine what measurements can be extracted.

For example, as will be appreciated by those skilled in the art, by appropriate sensor placement it is possible to measure any movement of the extremities, such as arm swing, elbow extension, neck rotation, knee extension, and postural sway. In one example, arm movement and elbow extension may be measured by placing one sensor on the shoulder, one on the elbow, and one on the wrist (on each side). In another example, leg movement and knee extension may be measured by placing one sensor on the ankle, one on the knee, and one on the hip (on each side).

The following discussion will focus on gait analysis, with two sets of paired sensors employed on the feet of the subject. However, it will be appreciated that the invention is not restricted to gait analysis or to the particular configuration of sensors shown in FIG. 1.

In the discussion below, u₁ and u₂ are on the right shoe 14 and u₃ and u₄ are on the left shoe 16. However, it will be appreciated that left and right can be interchanged, with consequential changes to the labelling of the sensor units. The first sensor unit (u₁ or u₃) may be positioned flat on the front toe area and the other (either u₂ or u₄) vertically oriented on the heel of the shoe. Therefore, the distance between the front of the two shoes (u₁ and u₃), is labelled as u̅₁̅u̅₃̅.

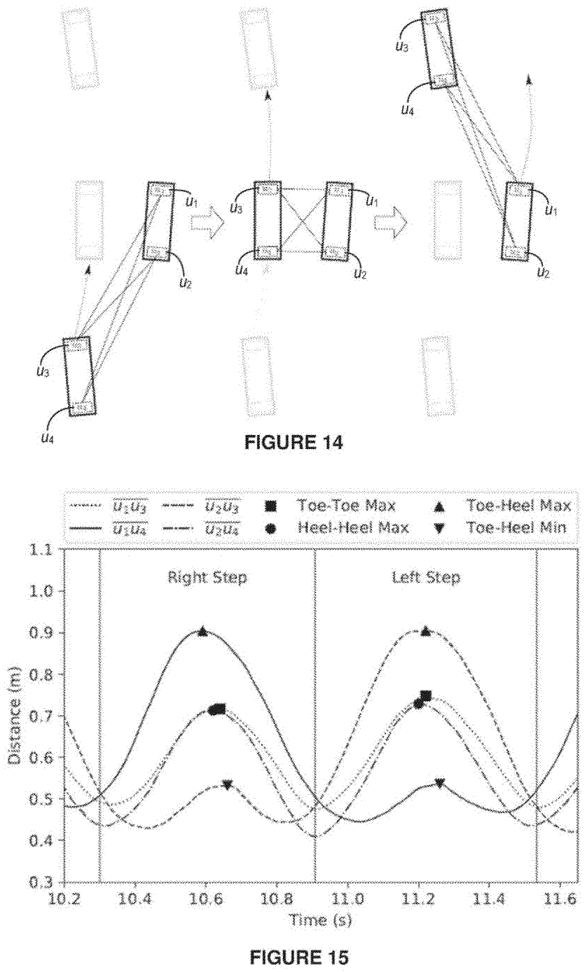

Sensors on either foot 14, 16 are a fixed distance away from each other (usually L the length of the shoe), and as a result these distances are not measured during gait analysis. These four measurement distances u̅₁̅u̅₃̅, u̅₁̅u̅₄̅, u̅₂̅u̅₃̅, u̅₂̅u̅₄̅ and the fixed length L make up a rigid irregular quadrilateral between the feet (see FIG. 14). The polygon defined by these measurements enables calculation of the step (and stride) length and width, and the placement of the shoes relative to each other.

The sensor units may individually or collectively transmit the following data to an external computing device, such as mobile device 12, at a sampling rate of 100 Hz:
Timestamp in milliseconds (t)
3 axes of Acceleration (aₓ, a_{y}, a_{z})
3 axes of Rotation (gₓ, g_{y}, g_{z})
2 distance measurements (u̅₁̅u̅₃̅ and u̅₁̅u̅₄̅ or u̅₂̅u̅₃̅ and u̅₂̅u̅₄̅)
Sensor temperature (Tₓ)

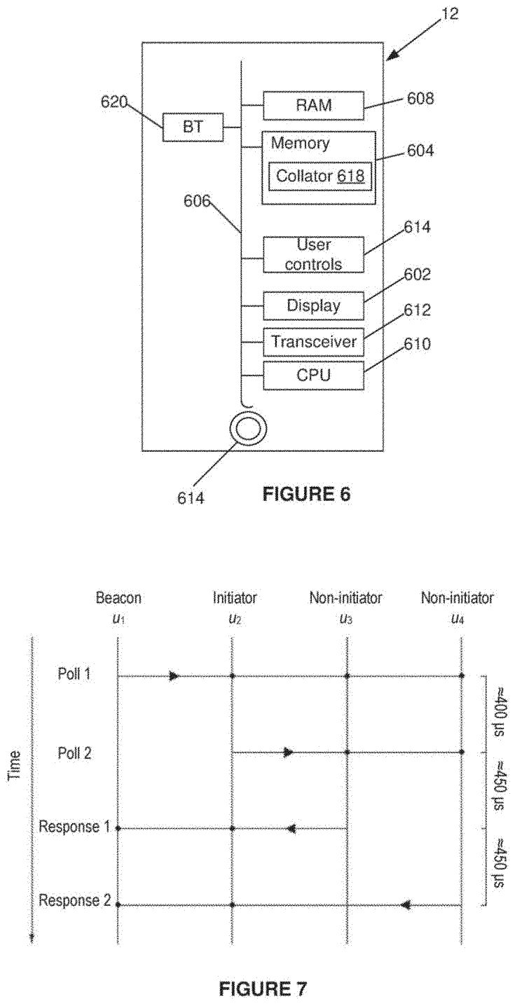

The mobile device 12 may execute a collator application 618 (FIG. 6). Collator application 618 may connect to and control the sensor units via their Bluetooth interfaces 34. In certain embodiments, a gait analysis session may be initiated at the collator application 618, which starts recording data from the sensor units. The collator application 618 may decode and save the raw data for later processing.

For best line of sight, the UWB aerials 26 of each sensor may be pointed inwards towards the other shoe. As the back of the heel has been found to be an optimum place to position an IMU for gait measurement, acceleration and angular speed measurements may be limited to the IMU sensors 22 on the back of the heels (sensor units u₂ and u₄). The orientation of the rear IMU sensor and its channels are illustrated in FIG. 4.

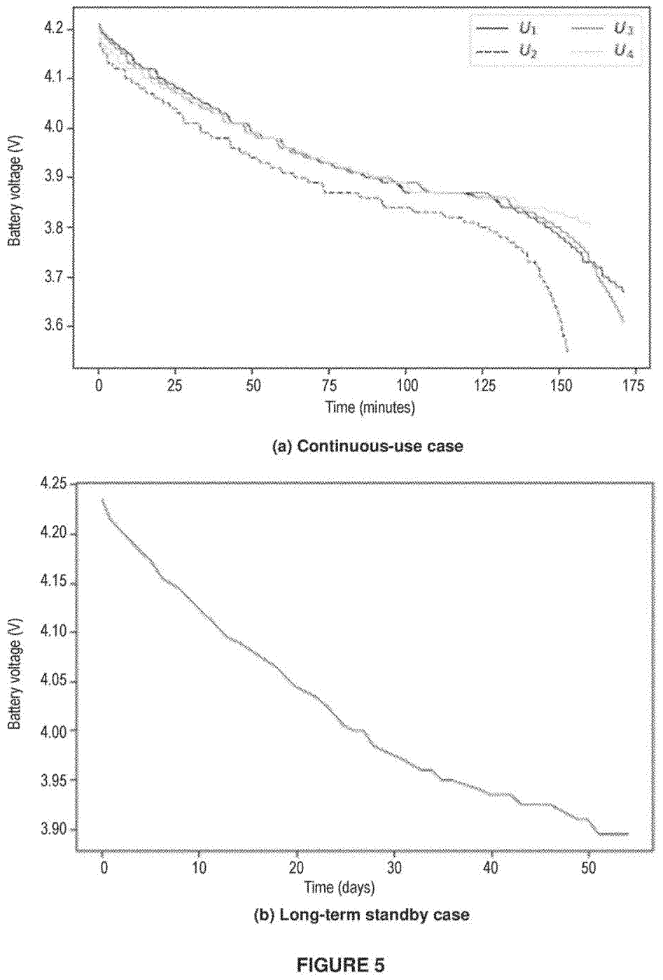

It has been found that the system 10 can run for up to 2.5 hours under continuous operation, as can be seen in FIG. 5a. In long-term standby mode, as can be seen in FIG. 5b, the sensors of system 10 can last at least fifty days.

### Mobile Computing Device 12

FIG. 6 is a block diagram showing an exemplary architecture of a computing device 12. The device 12 may be a mobile computer device such as a smart phone, a tablet, a personal data assistant (PDA), a palm-top computer, and multimedia Internet enabled cellular telephones. For ease of description, the mobile computer device 12 is described below, by way of non-limiting example, with reference to a mobile device in the form of an iPhone™ manufactured by Apple™, Inc., or one manufactured by LG™, HTC™ and Samsung™, for example.

As shown, the mobile computer device 12 includes the following components in electronic communication via a bus 606:
(a) a display 602;
(b) non-volatile (non-transitory) memory 604;
(c) random access memory (“RAM”) 608;
(d) N processing components 610;
(e) a transceiver component 612 that includes N transceivers;
(f) user controls 614; and
(g) a Bluetooth (e.g., BLE-compatible) module 620.

Although the components depicted in FIG. 6 represent physical components, FIG. 6 is not intended to be a hardware diagram. Thus, many of the components depicted in FIG. 6 may be realised by common constructs or distributed among additional physical components. Moreover, it is certainly contemplated that other existing and yet-to-be developed physical components and architectures may be utilised to implement the functional components described with reference to FIG. 6.

The display 602 generally operates to provide a presentation of content to a user, and may be realised by any of a variety of displays (e.g., CRT, LCD, HDMI, micro-projector and OLED displays).

In general, the non-volatile data storage 604 (also referred to as non-volatile memory) functions to store (e.g., persistently store) data and executable code.

In some embodiments for example, the non-volatile memory 604 includes bootloader code, modem software, operating system code, file system code, and code to facilitate the implementation components, known to those of ordinary skill in the art, which are not depicted nor described for simplicity. For example, the non-volatile memory 604 may contain a collator application 618.

In many implementations, the non-volatile memory 604 is realised by flash memory (e.g., NAND or ONENAND memory), but it is certainly contemplated that other memory types may be utilised as well. Although it may be possible to execute the code from the non-volatile memory 604, the executable code in the non-volatile memory 604 is typically loaded into RAM 608 and executed by one or more of the N processing components 610.

The N processing components 610 in connection with RAM 608 generally operate to execute the instructions stored in non-volatile memory 604. As one of ordinarily skill in the art will appreciate, the N processing components 610 may include a video processor, modem processor, DSP, graphics processing unit (GPU), and other processing components.

The transceiver component 612 includes N transceiver chains, which may be used for communicating with external devices via wireless networks. Each of the N transceiver chains may represent a transceiver associated with a particular communication scheme. For example, each transceiver may correspond to protocols that are specific to local area networks, cellular networks (e.g., a CDMA network, a GPRS network, a UMTS network), and other types of communication networks.

The mobile computer device 12 can execute mobile applications, such as the collator application 618. The collator application 618 could be a mobile application, web page application, or computer application. The collator application 618 may be accessed by a computing device such as mobile computer device 12, or a wearable device such as a smartwatch.

It should be recognised that FIG. 6 is merely exemplary and in one or more exemplary embodiments, the functions described herein may be implemented in hardware, software, firmware, or any combination thereof. If implemented in software, the functions may be transmitted or stored as one or more instructions or code encoded on a non-transitory computer-readable medium 604. Non-transitory computer-readable medium 604 includes both computer storage medium and communication medium including any medium that facilitates transfer of a computer program from one place to another. A storage medium may be any available medium that can be accessed by a computer.

### Device Synchronisation and Ranging Protocol 1000

To describe the motion of the subject's feet during use of the system 10, a high sampling rate is advantageous to increase accuracy. Embodiments of the system 10 may record IMU and UWB measurements on all four sensors at 100 Hz, time-locked to within a few hundred μs. However, this high sampling rate needs to be balanced with the power efficiency concerns of any wearable technology. To achieve better power efficiency, it is advantageous for the four sensors to know precisely when they should turn on their UWB radios 26 for any communication required.

In view of the above, the system 10 may implement a synchronisation process. In one example, each UWB sensor 24 may be configured to send an interrupt at regular intervals (e.g., every 10 ms for a sampling rate of 100 Hz) to corresponding processor 20. Alternatively, the interrupt may be initiated at the processor 20. The processor 20 of only one of the sensor units, such as sensor unit u₁, may be configured to transmit a synchronisation beacon message to each of the other sensors u₂, u₃ and u₄.

The designation of a sensor unit as the synchronisation beacon unit may be controlled by mobile device 12. In other embodiments, the designation of a sensor unit as the beacon unit may be negotiated among the sensor units. For example, a sensor synchronisation process may include each sensor unit waiting for a random length of time after start-up, and then transmitting a first signal from its UWB sensor 24. The first sensor unit to transmit becomes the beacon, the second to transmit becomes the non-beacon initiator, and third and fourth are non-initiators. In case of a conflict where any two sensor units transmit at the same time, an exponential backoff approach may be implemented, in which all units may give up their designations, and then wait a longer random amount of time than before to start the synchronisation protocol again.

The synchronisation beacon message may be transmitted after a fixed delay after the interrupt. The synchronisation beacon message can be used by the other sensor units u₂, u₃, u₄ to move their interrupt timings in-line with the beacon sent by sensor unit u₁. Over a few hundred milliseconds, it has been found that all devices become locked to this beacon pulse to within μs. Therefore, the timings of any potential ranging protocol are known to all of the devices, meaning that the UWB radios 26 are turned on or off as required. This allows the system 10 to reduce its power consumption and improve efficiency by only turning on the UWB radios 26 approximately 20% of the time.

With reference to FIGS. 7, 8, 9 and 10, an exemplary ranging protocol 1000 will now be described. In the following discussion, certain sensor units will be referred to as “initiators”, and others as “non-initiators”. Initiators begin the ranging protocol by sending poll (request) messages, and non-initiators reply to these requests. The initiator that starts the protocol is the beacon as it will broadcast (and hence dictate) the current time to all other devices.

In general, a single-sided two-way ranging protocol 1000 may include:
a first (initiator) sensor unit (e.g., u₁) transmitting a first poll signal to each other sensor unit (e.g., u₂, u₃, u₄); and
one or more of the other (non-initiator) sensor units (e.g., u₃ and u₄) transmitting a response signal, or response signals, to the first sensor unit u₁, each said response signal including a difference between a time of receipt of the first poll signal at the respective other sensor unit and a time of transmission of the response signal.

The timing differences reported by the non-initiator sensor units allow the initiator sensor unit to determine the relative motion between it and each non-initiator sensor unit.

In the example described below, the sensors on the right shoe (u₁ and u₂) behave as initiators and the other two (u₃ and u₄) behave as non-initiators. It will be appreciated that the roles of initiators and non-initiators can be switched as desired, e.g. under control of mobile device 12, or can be negotiated between the sensor units at start-up, as discussed above.

Each sensor unit samples its onboard IMU 22 and transmits a Bluetooth packet within each interrupt interval of 10 ms. Accordingly, there is a very limited time budget of 2 ms within each interrupt interval to implement the entire UWB ranging protocol 1000. Were it not for these time constraints, the ranging protocol could be run at 200-500 Hz, albeit with a much higher power consumption.

Conventionally, UWB ranging uses a symmetric double-sided two-way ranging protocol, where the protocol is structured to remove the errors caused by the different relative speeds between device oscillators/clocks. However, this approach requires more messages and a much larger time budget. Accordingly, embodiments of the present invention use a custom single-sided two-way ranging protocol with poll and response messages. In certain embodiments, only the initiators compute a distance measurement. This enables further speedup of the protocol.

With reference to FIGS. 7 and 10, an exemplary protocol 1000 involves four messages, with approximate timings shown in FIG. 7.

In a first step 1010, the first sensor unit u₁ (the beacon) transmits a first poll signal, denoted as Poll 1 in FIG. 7, to the second (initiator), third (non-initiator) and fourth (non-initiator) sensor units u₂, u₃ and u₄. Poll 1 may be a 7 byte message containing a beacon time, to which the other sensors synchronise as discussed above, and requesting a range.

In a second step 1020, the second sensor unit u₂ transmits a second poll signal, denoted as Poll 2 in FIG. 7, to the third (non-initiator) and fourth (non-initiator) sensor units u₃ and u₄. Poll 2 may be a 3 byte message requesting a range.

In a third step 1030, the third sensor unit u₃ transmits a first response message, denoted as Response 1 in FIG. 7, to the first (beacon) and second (initiator) sensor units u₁ and u₂. Response 1 may be an 11 byte message containing the time in 15.65 picosecond increments (15.65 μs being the time resolution of the oscillators of the UWB sensors 24) between the time of arrival of the received poll messages from u₁ and u₂, and the expected transmission time of this response message.

In a fourth step 1040, the fourth (non-initiator) sensor unit u₄ transmits a second response message, denoted as Response 2 in FIG. 7, to the first (beacon) and second (initiator) sensor units u₁ and u₂. Response 2 may be an 11 byte message containing the time in 15.65 picosecond increments between the time of arrival received poll messages from u₁ and u₂, and the expected transmission time of this response message.

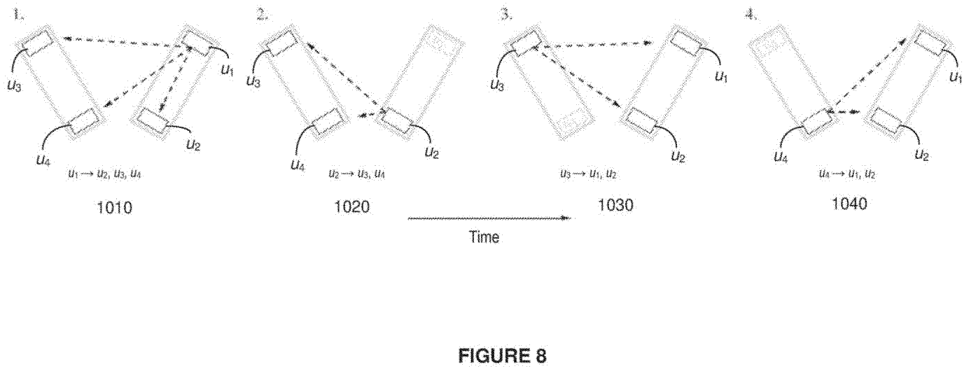

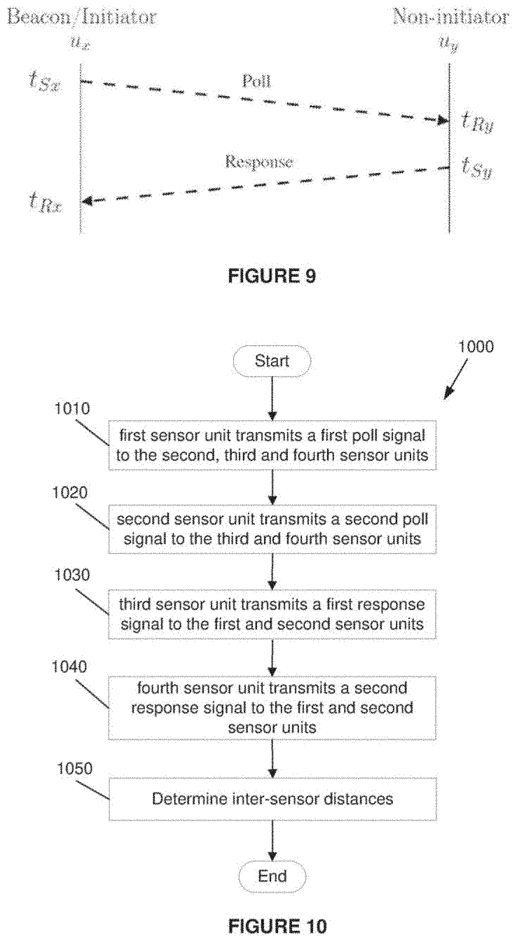

The ordering of these transmission steps can be seen in FIGS. 7 and 8. These Poll and Response messages provide the system 10 with the required timestamps for ranging as can be seen in FIG. 9. The protocol 1000 takes approximately 1.8 milliseconds, and is performed 100 times a second.

After receiving a response, at step 1050, the initiators u₁ and u₂ calculate the duration from sending a poll message to receiving the response message (t_{R}_{ₓ}−t_{S}_{ₓ}) and then these timestamps are used for calculating the time-of-flight (t_{x,y}) between uₓ and u_{y}, and as a result the distance between them. The formula is

$${t}_{x , y} = \frac{( {t}_{{R}_{x}} - {t}_{{S}_{x}} ) - ( {t}_{{R}_{y}} - {t}_{{S}_{y}} )}{2}$$

and the distance between sensor uₓ and u_{y} is therefore,

u̅ₓ̅u̅_̅{̅y̅}̅=ct_{x,y},

where c is the speed of light. Whilst the speed of a UWB transmission through air is a little slower than c, it is only slower by approximately 0.03%, and this difference is negligible.

Advantageously, the distances between the sensors may be computed only by the initiator sensor units. This reduces the time required to execute the ranging protocol 1000.

These time differences are made with respect to the different oscillators on each device and as a consequence they may not be exactly in sync. Additionally, there may be errors associated with antenna delay and manufacturing differences in the DWM1000 chip, and as such the UWB ranging sensor may need to be calibrated.

For example, a UWB calibration process may be implemented to correct for sources of error in ranging estimation that arise from temperature variation, including antenna delay and differences in oscillator drift. Antenna delay is the internal delay of the chip, and it is determined by the differences in the shape of the aerial and the device temperature. The ranging error can be as much as 2.15 mm per degree centigrade. If a single-sided two-way protocol 1000 is used, oscillator drift can be problematic. The oscillator has a warm up time and is also affected by device temperature.

Calibration is performed by comparing known distances with UWB measurements. A linear model is fitted to the calibration data to compensate for any error associated with the geometry of the sensor arrangement, the antenna delay, and oscillator differences. The calibrated measurement between device x and y may be expressed as:

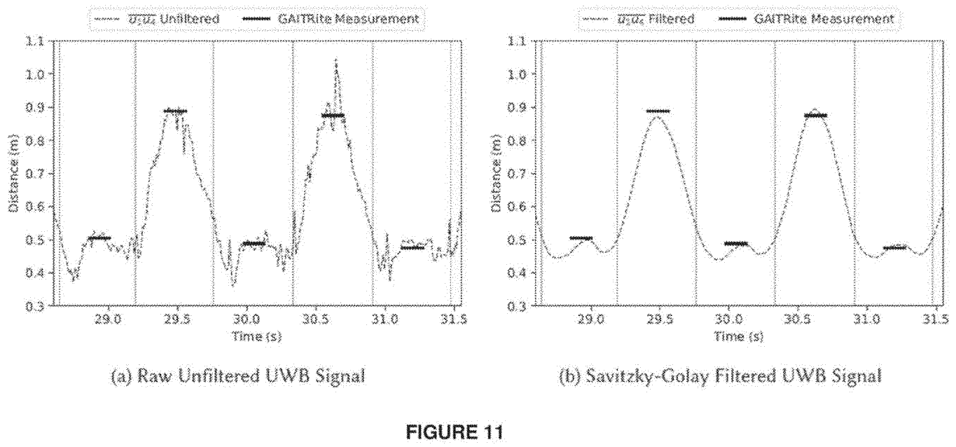

u̅ₓ̅u̅_̅{̅y̅}̅_̅{̅_̅{̅c̅a̅l̅i̅b̅}̅}̅=ρ₀u̅ₓ̅u̅_̅{̅y̅}̅+ρ₁(Tₓₘₐₓ−Tₓ)+ρ₂(T_{ymax}−T_{y})+ρ₃,

where Tₓ is the temperature of device x, Tₓₘₐₓ is the maximum temperature device x reaches at steady state and ρ₀, ρ₁, ρ₂, and ρ₃ are model parameters. UWB walking data corrected using this fitted model can be seen in comparison with the direct measurements from the GAITRite in FIGS. 11a and 11b. In the second peak of FIG. 10a, we can see a spike at the apex, which is likely due to the sensors losing line of sight due to the positioning of the feet. However, this effect is lessened by the use of a Savitzky-Golay filter as can be seen in FIG. 11b. Note that in both the raw unfiltered and filtered figures, the maximum and minimum peaks line up with the GAITRite measurements.

Inertial sensor calibration may also be performed. For example, an MPU6050 IMU uses MEMS (Microelectromechanical systems) for the accelerometers and gyroscopes. As these are physical systems, they have slight manufacturing differences, and therefore these MEMS units vary from one another, and thus require individual calibration, primarily to correct the “zero points” for each axis. A factory calibration is done which sets trim values, but each unit may be recalibrated as this factory calibration is not always reliable.

To calibrate each unit 22, they may be held in a particular orientation such that the accelerometer directions for two channels are perpendicular to, and the other channel is co-linear with, the gravitational field of the earth. The units are operated for a sufficient length of time (e.g. 10 minutes) to achieve a constant temperature, and then tens of thousands of readings are made using MPU Offset Finder code (www.fenichel.net). This program resets a window of trim offsets until each axis moves to the correct values for this orientation. For example, with the Z axis (perpendicular to the IC) pointing down, it should record exactly 1 g. Trim offsets are changed until they read (aₓ=0, a_{y}=0, a_{z}=1, gₓ=0, g_{y}=0, g_{z}=0). The final trim offset values are preloaded into the device each time it is used.

### Motion Metric Determination Method 1600

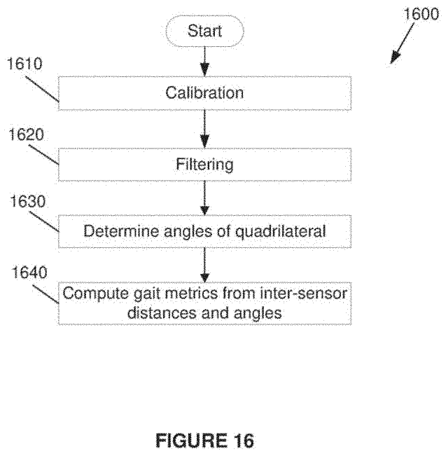

An embodiment of a motion metric determination method 1600 will now be described (FIG. 16). The motion metric determination method 1600 makes use of TOF ranging data, optionally in combination with inertial sensor data. The example method described below relates to gait metrics, but it will be understood that other types of motion metric, such as arm swing, may be determined by changing the placement of the sensor units on the subject, as mentioned above.

In general, the method 1600 may include determining (e.g., by a peak detection process) one or more stationary points, such as local maxima, local minima, or points of inflection, of a curve defined at least partly by the TOF ranging data and/or the inertial sensor data, and computing at least one motion metric based on the one or more stationary points.

Firstly, in order to interpret the signals coming from the UWB sensors 24, we consider the geometry of the arrangement of the sensors u₁, u₂, u₃, u₄, and how they change over time.

During the double support time (both feet on the ground), some assumptions can be made about the meanings of the UWB measured distances. In FIG. 13, it can be seen that the Heel-Heel distance is given by u̅₂̅u̅₄̅, and the Toe-Toe distance u̅₁̅u̅₃̅.

The maximum Toe-Heel distance (Toe-Heel Max) in this example is given by u̅₂̅u̅₃̅ and the minimum Toe-Heel distance (Toe-Heel Min) is given by u̅₁̅u̅₄̅. This is because it is a left step and these will be reversed in the right step. Therefore, the maximum and minimum Toe-Heel points are a good proxy for step indicators.

Now, consider the change in these measurements in motion. Unlike the GAITRite, the system 10 has the potential to measure the step width whilst the foot is in the air. In FIG. 14, the general behaviour of the measured step lengths is shown. Note that the minimum Toe-Heel distance and maximum Toe-Heel distance swap after every step. Furthermore, in mid step, it can be seen that all of the measurements get smaller as the left foot approaches the other. This minimum is a good proxy for the midway point of the stride.

Turning now to FIG. 16, at step 1610, a calibration operation is performed before performing any calculations on the UWB signals. The calibration operation 1610 may include using temperature measurements reported by the sensors, and a UWB calibration function (as described above), to correct for measurement error.

Next, at step 1620, the signal may be filtered, for example using a Savitzky-Golay filter. This filter is advantageous as it does not greatly distort the shape of the signal, but still smooths out some of the noise. A continuous wavelet transformed-based peak detection algorithm may be used to find the peaks in all four signals. The four signals are the respective estimated distances u̅ₓ̅u̅_̅{̅y̅}̅ determined by the ranging protocol 1000 as a function of time. This set of four peaks represents the best estimate of the foot positioning at every step. A sample of two steps is shown in FIG. 15. Note, as expected, the Heel-Toe Max and Heel-Toe Min swap between u̅₁̅u̅₄̅ and u̅₂̅u̅₃̅.

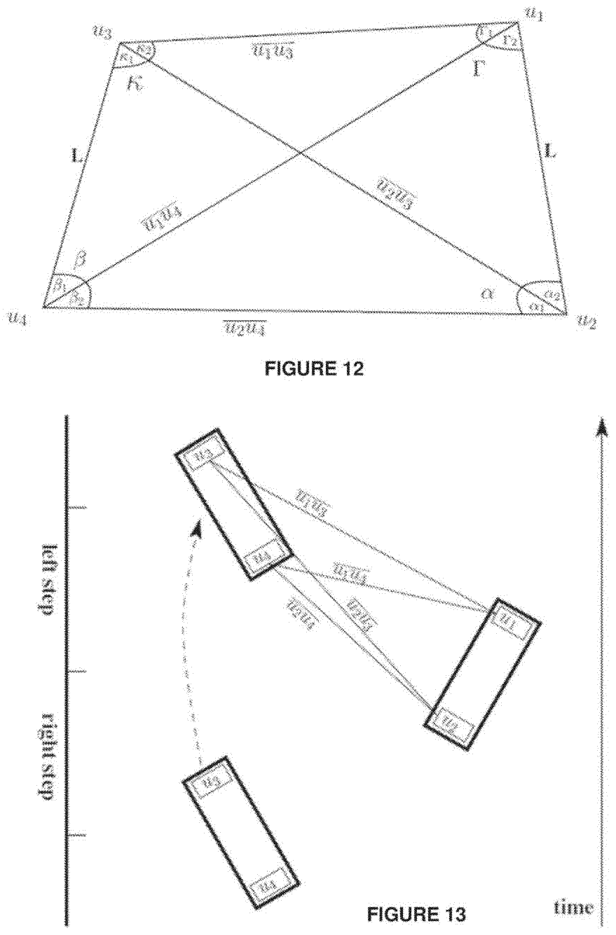

At step 1630, the internal angles of the irregular quadrilateral present between the two feet (FIG. 12) are determined. These can all be found using the cosine rule, and are shown below.

$$β = arccos \frac{{\overline{{u}_{2} {u}_{4}}}^{2} + {L}^{2} - {\overline{{u}_{2} {u}_{3}}}^{2}}{2 \overline{{u}_{2} {u}_{4}} L} , κ = arccos \frac{{\overline{{u}_{1} {u}_{3}}}^{2} + {L}^{2} - {\overline{{u}_{1} {u}_{4}}}^{2}}{2 \overline{{u}_{1} {u}_{3}} L} , Γ = arccos \frac{{\overline{{u}_{1} {u}_{3}}}^{2} + {L}^{2} - {\overline{{u}_{2} {u}_{3}}}^{2}}{2 \overline{{u}_{1} {u}_{3}} L} , α = arccos \frac{{\overline{{u}_{2} {u}_{4}}}^{2} + {L}^{2} - {\overline{{u}_{1} {u}_{4}}}^{2}}{2 \overline{{u}_{2} {u}_{4}} L} .$$

Having determined the inter-sensor distances and the internal angles of the quadrilateral, it is possible to calculate some important gait metrics, at step 1640.

First, the step time (Stpₜ) is defined as the difference of two consecutive alternating Heel-Toe maximums, and the stride time (Strₜ) is the sum of two consecutive step times. Stance time can be calculated based on the proportion of time that the UWB signal spends above an empirically defined threshold (time spent at the top of the Heel-Toe peak), and swing time is just the rest of that proportion. Cadence can be calculated by counting the number of Heel-Toe maximums in a fixed duration. The step length is defined as the component of u̅₂̅u̅₄̅ in the direction of walking,

Stpₗ=u̅₂̅u̅₄̅ cos β₂ or u̅₂̅u̅₄̅ cos α₁,

for the right and left steps respectively, where the angles β₂ and α₁ are defined as

$${β}_{2} = arccos \frac{{\overline{{u}_{1} {u}_{4}}}^{2} + {\overline{{u}_{2} {u}_{4}}}^{2} - {L}^{2}}{2 \overline{{u}_{1} {u}_{4}} \overline{{u}_{2} {u}_{4}}} , \\ and \\ {α}_{1} = arccos \frac{{\overline{{u}_{2} {u}_{3}}}^{2} + {\overline{{u}_{2} {u}_{4}}}^{2} - {L}^{2}}{2 \overline{{u}_{2} {u}_{3}} \overline{{u}_{2} {u}_{4}}} .$$

The stride length Strₗ is defined as two consecutive step lengths. The step width is defined as the distance between the two mid points of the feet which by geometry is,

$$S t {p}_{w} = \frac{\overline{{u}_{1} {u}_{3}} + \overline{{u}_{2} {u}_{4}}}{2}$$

Furthermore, we can calculate the stride velocity as

$$S t {r}_{V} = \frac{S t {r}_{l}}{S t {r}_{t}} .$$

Additionally, with these angles it is possible to calculate the positioning of the feet (relative to each other).

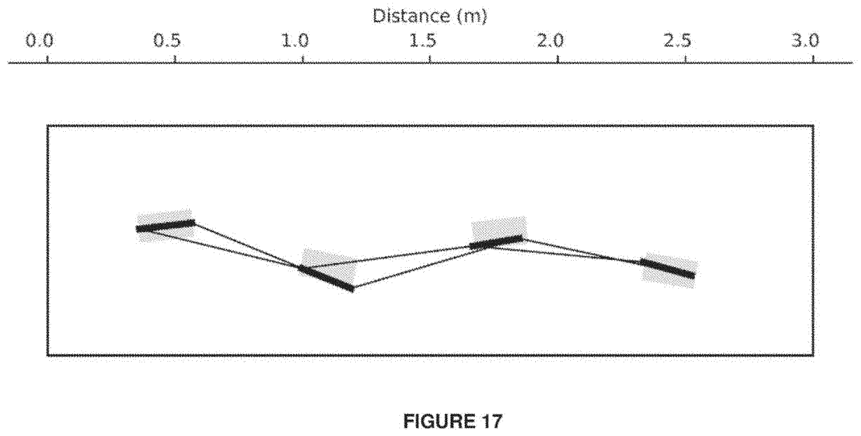

The foot placement determined using system 10 and methods 1000, 1600 is compared with the foot placement of the GAITRite mat in FIG. 17, in which measurements determined by the system 10 are anchored spatially to the first foot of the GAITRite. FIG. 17 shows the plotted trajectory of three steps of a walk, where each irregular quadrilateral is taken from the Heel-Toe maximum point. The gray footprints in the figure show the GAITRite foot positioning. The thick black lines represent the best estimation of foot placement by system 10. Even though the angles calculated between these measurements are at their least accurate at this point (due to some sensors not having direct line of sight) it is still possible to calculate these step locations. Note that the walk is straight and does not drift to either side, contrary to what can be seen in IMU-based sensor measurements. Importantly, all of these calculations are low complexity enough that they could be run on embedded hardware.

Gait Metrics from IMU Data

Optionally, as part of step 1640, one or more metrics can be determined from the inertial sensor data.

For example, the raw IMU data may be linearly interpolated, re-sampled at 1000 Hz, and then low-pass filtered with a cutoff of 10 Hz (to remove noise), as is implemented in some known IMU-based studies. With this cleaned and interpolated data, we now turn to the problem of estimating stride length. As system 10 does not include a magnetometer, it is very difficult to orientate the sensors with respect to each other, and therefore does not consider the estimation of step length using only IMUs. We therefore use a simple (and low computational cost) zero-velocity update double integration method, based on using the gyroscope to compensate for the change in orientation of the sensor during walking. These methods can be run on embedded hardware.

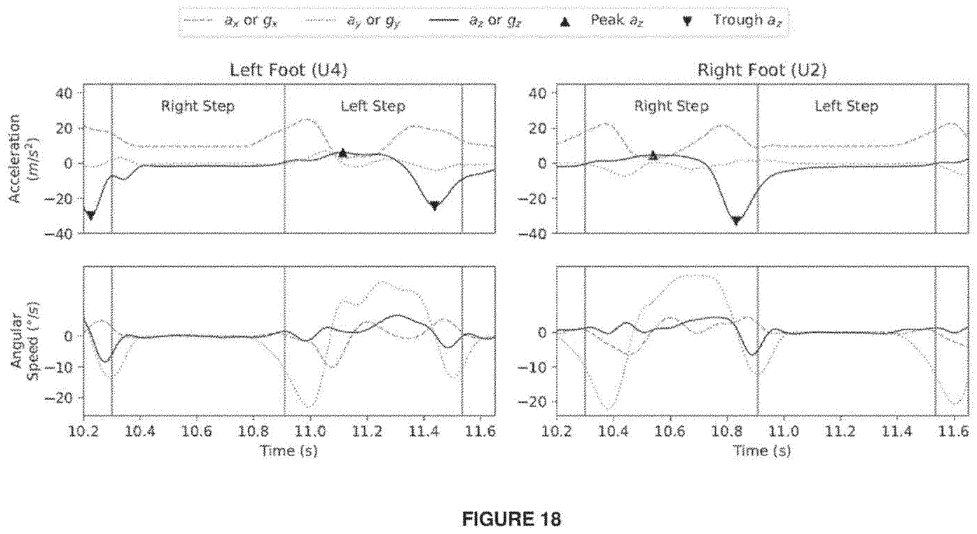

As the IMU sensors 22 are oriented such that all three accelerometers are approximately in line with the three planes of the body, we can use the following general definitions: aₓ is the acceleration up-and-down, a_{y} is the acceleration left-to-right, and a_{z} is the acceleration back-to-front. FIG. 18 shows an example of the IMU data recorded during walking, and it is taken from the same two steps as in FIG. 15. In general, it can be seen that most of the acceleration occurs in the up-down (aₓ) and back-front (a_{z}) directions, and this intuitively makes sense in the context of walking. We can also see the dominant rotation around the left-right axis (g_{y}). This is the ankle rotating during a walk.

The first stage of the zero-velocity update double integration method involves finding the peaks and troughs in a_{z}. These peaks are analogous to the toe-off and heel-strike events that occur during walking. The stride time can therefore be defined as the distance between two consecutive peaks. The stride length is defined as the double integral of the acceleration in the forward direction. However, due to the rotation of the sensor, the channels of acceleration cannot be used directly.

The first model involved using the gyroscope g_{y}, to compensate for the rotation of the ankle. Using this method, the acceleration in aₓ and a_{z} is combined into a vector based on the angle of rotation. The method only uses 3 axes of the IMU. Another approach is to use all 6 axes of the gyroscope and accelerometers to compensate for the 3D motion of the foot. As before these IMU readings are transformed from the IMU reference frame, into a global reference frame, this time using the method based on the Direction Cosine Matrix (a full description of this methodology can be found in W. Premerlani and P. Bizard, “Direction Cosine Matrix IMU: Theory”, Technical Report, 2009). Once these transformed values are found, the signal is integrated twice between the consecutive peak and trough, this being the stride length.

### Experimental Results

Walking data from twenty one healthy adults (between the ages of 21 and 35), were recorded concurrently with a GAITRite walking mat and with system 10. An overview of the dataset is shown in Table 1.

The GAITRite walking mat is capable of measuring stride time, step time, stride length, step length, stride width, and step width amongst others and is therefore a good choice to serve as a ground truth measurement and as a comparison to system 10. GAITRite claims a spatial resolution accuracy of ±1.27 cm.

Each subject walked on a walking track for more than 80 steps, split up into 15 “sessions”, defined as one walk over the mat. All data was collected with Institutional Review Board of [BLINDED] approval. This data was collected to mimic a standard gait assessment. Subjects were asked to walk a comfortable pace over the walking mat and after each session the subject could choose to rest. The GAITRite was synchronised with system 10 using NTP to a local NTP server. This data was collected over a two week period at [BLINDED].

TABLE 1

| Metric | Value |
|---|---|
| Number of Subjects | 21 |
| Number of Steps | 2091 |
| Number of Strides | 1820 |
| Male/Female | 10/11 |
| Age Range | 21-35 |
| Step time range (s) | 0.47-0.80 |
| Step length range (m) | 0.48-0.85 |
| Step width range (m) | 0.49-0.87 |
| Stride time range (s) | 0.96-1.51 |
| Stride length range (m) | 0.97-1.69 |
| Stride velocity range (m/s) | 0.67-1.74 |
| Cadence range (steps per minute) | 87.52-122.03 |
| Swing time range (s) | 0.33-0.56 |
| Stance time range (s) | 0.59-1.02 |

### UWB-Only Measurements

First we will look at the UWB-only methods for measuring gait metrics. Whilst there are undoubtedly some errors introduced due the simplification of the 3-dimensional nature of the physical system, this model performs well. Table 2 shows the root mean square error (RMSE), mean absolute error (MAE) and mean absolute percentage error (MAPE) of UWB measurements in comparison to the GAITRite. We can see across most metrics we are getting within 4-5% of the ground truth value. Additionally, we are measuring step width which is not a metric calculated by standard wearables. Despite the limitations of UWB measurement ranging technologies we are able to get accurate gait metrics.

TABLE 2
Comparison of UWB measurements vs. GAITRite

| Metric | RMSE | MAE | MAPE |
|---|---|---|---|
| Step Time (s) | 0.016 | 0.012 | 2.14% |
| Stride Time (s) | 0.016 | 0.011 | 1.01% |
| Step Width (m) | 0.041 | 0.033 | 4.85% |
| Step Length (m) | 0.041 | 0.032 | 4.75% |
| Stride Length (m) | 0.070 | 0.056 | 4.11% |
| Stride Velocity (m/s) | 0.067 | 0.051 | 4.23% |
| Cadence (steps/min) | 0.703 | 0.442 | 0.42% |
| Swing Time (s) | 0.029 | 0.022 | 5.17% |
| Stance Time (s) | 0.030 | 0.022 | 3.07% |

### IMU Methods

The simple IMU methods used herein do not perform as well as the UWB metrics. This is expected as these methods are low time-complexity, and relatively simplistic. Table 3 shows the root mean square error (RMSE), mean absolute error (MAE), and mean absolute percentage error (MAPE) of IMU measurements in comparison to the GAITRite. We can see the temporal measurements are very similar to the UWB methods, however the spatial metrics perform worse. These results are consistent with those from other researchers using foot-mounted 6-axes IMU methods. These IMU measurements though are not affected by line of sight as UWB measurements are and therefore we will now look at a simple fusion of the two methods.

TABLE 3
Comparison of IMU measurements vs. GAITRite

| Method | Metric | RMSE | MAE | MAPE |
|---|---|---|---|---|
| 3 & 6 Axes | Stride Time(s) | 0.056 | 0.022 | 1.96% |
| 3 Axes | Stride Length (m) | 0.132 | 0.105 | 7.96% |
| 6 Axes | Stride Length (m) | 0.140 | 0.111 | 8.42% |

### Simple Fusion

Although it performs worse overall, the IMU can be more accurate in measuring the stride length in a subset of our dataset. This is likely due to the UWB sensors temporarily losing line of sight during a narrow step (recall the spike in FIG. 11a). To take advantage of this, we combine our stride lengths using a linear sum of the length measurements from the two sensor types, the linear sum having the form:

Strₗ=v₀Strₗ^{UWB}+v₁Strₗ^{IMU}.

The coefficients v₀ and v₁ may be found using standard mathematical optimisation techniques, such as least squares regression, for example. In one example, v₀=0.82 and v₁=0.18.

We also try compensating for the slight difference in UWB stride length error between measuring the left and right strides by fitting this same model for the left and right strides independently. An approximate 20% improvement in accuracy by combining the two with these methods can be seen in Table 4.

TABLE 4
Simple Fusion of IMU and UWB

| Method | Metric | RMSE | MAE | MAPE |
|---|---|---|---|---|
| Linear | Stride | 0.064 | 0.050 | 3.72% |
| Combination | Length | | | |
| | (m) | | | |
| Linear | Stride | 0.063 | 0.050 | 3.70% |
| Combination | Length | | | |
| (Strides) | (m) | | | |

In summary, the system 10 can measure gait metrics that are not possible on such IMU-based wearables, and stable spatial readings of steps is also possible. It is apparent that a combination of IMU and UWB can give an increase in accuracy. Embodiments of the system 10 therefore combine these two technologies to provide the benefits of both IMU systems and walking mats at a fraction of the cost. The GAITRite walking mat costs in the order of tens of thousands of dollars, whereas a set of four sensors such as the one shown in FIG. 2 is less than five hundred dollars (even before large scale manufacture). The sensors can also be precisely synchronised between themselves, and allow for accurate foot placement/stance estimation. Unlike the GAITRite mat, embodiments of the present system can give direct measurements of foot movement through the whole gait cycle, even when the feet are in motion and above the mat. This measurement throughout the whole gait cycle allows direct measurement of gait parameters such as stride length (as one foot passes by the other) rather than estimation based on foot placement, as the GAITRite does.

In some embodiments of the motion analysis system and method, the TOF ranging sensor (such as UWB sensor) data and IMU data may be combined via a sensor fusion process. Accordingly, the inter-sensor distances and/or the one or more motion metrics may be determined based on a combination of the UWB data and IMU data.

### Extended Kalman Filter

In a first example of a sensor fusion process, the UWB data and IMU data may be combined via an extended Kalman filter.

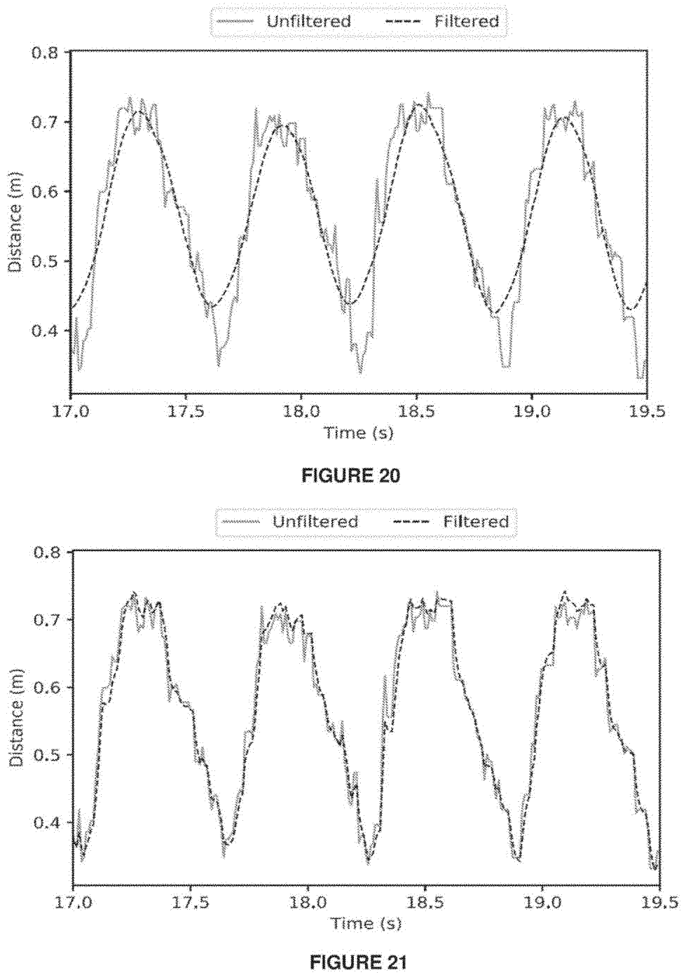

The use of traditional filtering methods can both time shift and change the shape of the raw signal as can be seen in FIG. 20. It can also be observed that the peaks of the signal are rounded, and that this does not accurately reflect the physical system. This is because during stance there should be a plateau, not a rounded peak as the feet are not moving and thus the measurements at this point are constant. In addition, the quality of the UWB measurements when the sensors have no line of sight, particularly at stance, may be compromised. However, the IMU data is not affected during this period and could be used to compensate for this introduced error.

Kalman Filtering is an algorithm to combine noisy measurements from multiple sensors to estimate system states more accurately. It is typically used in positioning or localisation systems such as those in commercial aircraft or drones. The EKF is the non-linear version of the Kalman Filter. In order to use EKF, the kinematic behaviour of the system is modelled.

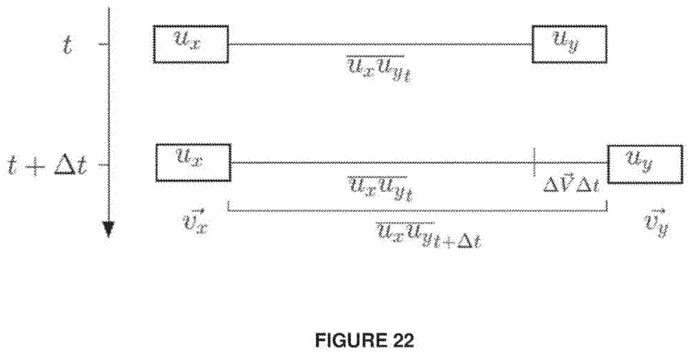

To motivate the present use of EKF, consider the physical system that is being measured. In system 10 there are four sensor units moving relative to each other. Initially, considering that if two sensors uₓ and u_{y} are moving apart with a velocity of ΔV, after a small amount of time (Δt) they will be ΔV Δt further apart as can be seen in FIG. 22. Conversely, if they are moving closer together this difference is −ΔV Δt. Therefore, there are two ways of measuring the displacement between these sensors, first using a ranging measurement u̅ₓ̅u̅_̅{̅y̅}̅, and second using the relative velocity of the devices. Assuming that the distance between uₓ and u_{y}, u̅ₓ̅u̅_̅{̅y̅}̅ₜ, and their relative velocity, is known, after a small amount of time Δt, the next distance measurement u̅ₓ̅u̅_̅{̅y̅}̅_{t+Δt} can be estimated as follows.

u̅ₓ̅u̅_̅{̅y̅}̅_{t+Δt}=u̅ₓ̅u̅_̅{̅y̅}̅ₜ+(\vec{v}ₓ−\vec{v}_{y})Δt.

However, the velocity cannot be measured directly as only an IMU is being used, therefore we define the velocity of uₓ as the cumulative acceleration:

\vec{v}_{x,t+Δt}=\vec{v}_{x,t}+\vec{a}_{x,t}Δt,

and assuming over this short amount of time that the acceleration remains constant, the acceleration after Δt must be

\vec{a}_{x,t+Δt}=\vec{a}_{x,t}.

Accordingly, there are two ways to estimate distances in the system 10. The following rules can be defined for all four UWB measurements that we have in the system:

u̅₁̅u̅₃̅_{t+Δt}=u̅₁̅u̅₃̅ₜ+(\vec{v}₁−\vec{v}₃)Δt

u̅₁̅u̅₄̅_{t+Δt}=\vec{u₁u₄}ₜ+(\vec{v}₁−\vec{v}₄)Δt

\vec{u₂u₃}_{t+Δt}=u̅₂̅u̅₃̅ₜ+(\vec{v}₂−\vec{v}₃)Δt

u̅₂̅u̅₄̅_{t+Δt}=u̅₂̅u̅₄̅ₜ+(\vec{v}₂−\vec{v}₄)Δt

With these equations in place, an EKF model can be formulated. In some embodiments, only one of the acceleration axes may be used, specifically the direction that is collinear with the direction of walking. The presently described embodiment of the system 10 has three measurements it can make at every iteration. These are the ranging estimate between uₓ and u_{y}, the acceleration of uₓ, and the acceleration of u_{y}, as defined below.

$${z}_{k} = [ \begin{matrix}{\overline{{u}_{x} {u}_{y}}}_{k} \\ {a}_{x , k} \\ {a}_{y , k}\end{matrix} ]$$

There are five internal states of the model: the distance (d_{xy,k}) between uₓ and u_{y}, and the velocities and accelerations of the two sensors.

$${x}_{k} = [ \begin{matrix}{d}_{xy , k} \\ {v}_{x , k} \\ {v}_{y , k} \\ {a}_{x , k} \\ {a}_{y , k}\end{matrix} ]$$

Assuming constant acceleration, a transition matrix can be defined as

$${x}_{k + Δ t} = f ( {x}_{k} ) = [ \begin{matrix}{d}_{xy , k} + s g n ( {a}_{x , k} - {a}_{y , k} ) ( {v}_{x , k} - {v}_{y , k} ) Δ t \\ {v}_{x , k} + {a}_{x , k} Δ t \\ {v}_{y , k} + {a}_{y , k} Δ t \\ 1 \\ 1\end{matrix} ]$$

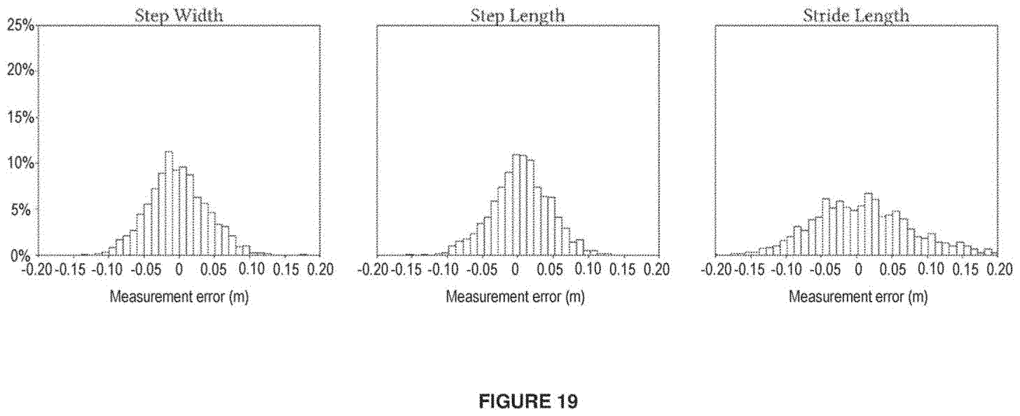

To implement this model, the python library FilterPy (https://github.com/rlabbe/Kalman-and-Bayesian-Filters-in-Python/) can be used. The EKF parameters such as Q and R matrices were found through experimentation. For each distance (u̅₁̅u̅₃̅, u̅₁̅u̅₄̅, u̅₂̅u̅₃̅ and u̅₂̅u̅₄̅) an independent EKF was run. The filtered signal was used similarly to the baseline method as can be seen in the algorithm below. The algorithm accuracy can be seen in Table 5 and in FIG. 19. It can be seen that even for step length and stride length, it performs only marginally better than the method described above. However, this method has many other upsides.

Algorithm 2:
CALCULATING GAIT METRICS (EKF)

| | |
|---|---|
| 1 | Input: Time T, [symbol] , [symbol] , [symbol] , [symbol] |
| 2 | Filter signals using the Extended Kalman filter |
| 3 | Find all peaks [symbol] in [symbol] |
| 4 | Calculate Step and Stride length/width by estimating |
| | internal angles using [symbol] |

This method gives an interpretable model, as the measurements made in between peaks are preserved in the filtering process, as well as the expected plateau at the top of each peak. This filter can also be compared with the method that does not make use of sensor fusion, as can be seen in FIG. 21. Note the plateau near the top of the peak and that the signal has not changed in shape. It also correctly finds the trough points which are useful for directly measuring stride length as well as the distance of the feet during the full walking motion.

TABLE 5
Comparison of EKF UWB measurements vs. GAITRite.

| Metric | RMSE | MAE | MAPE |
|---|---|---|---|
| Step Width (m) | 0.037 | 0.030 | 4.37% |
| Step Length (m) | 0.037 | 0.028 | 4.25% |
| Stride Length (m) | 0.075 | 0.058 | 4.34% |

### Support Vector Regression

In a second example of a sensor fusion process, the UWB data and IMU data may be combined via Support Vector Regression (SVR). In some embodiments, three different models may be used to calculate three different spatial gait metrics.

Before using SVR, we preprocessed and organised the raw IMU and UWB data. Specifically,

(1) the UWB measurements were calibrated as discussed above, and filtered using a Savitsky-Golay filter (though it will be appreciated that other smoothing filters, such as a Kalman filter or Butterworth filter may also be used);

(2) the IMU measurements were calibrated as discussed above;

(3) the step peaks and stride peaks in the UWB measurements were identified; and

(4) all IMU and UWB measurements were truncated to ±300 ms around each step peak, or either side of the stride peaks.

Then for each step and stride, we extracted a total of 113 features from the IMU data and UWB data of all four sensors. These features included the min, max, mean and standard deviation of all 24 channels of IMU data (6 channels for each sensor) and 4 channels of UWB data. We also use as a feature the baseline estimate of our metrics (i.e., the estimates obtained in step 1640 of FIG. 16), for example the baseline estimate of step width. We then used feature normalisation and used the libsvm-based scikit-learn SVR function to implement the SVR model.

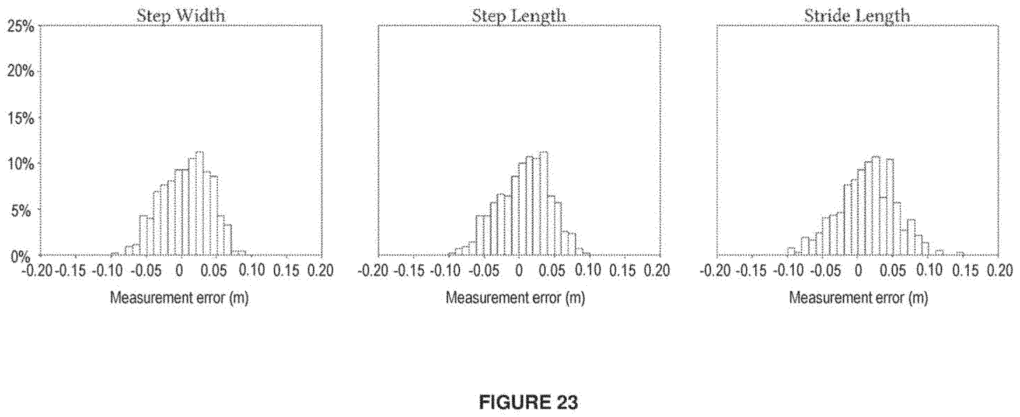

To find the optimum SVR kernel function, we used 10-fold cross validation and the steps dataset is divided into ten partitions such that a similar percentage of the amount of steps from each participant is in each partition. Through experimentation we found that, for step width and length, a SVR using the sigmoid kernel performed the best. For stride length, we found that a model with a linear kernel outperforms the sigmoid. The results of the SVR model estimations are shown in Table 6 and in FIG. 23. Note that compared to the baseline and EKF model, the SVR model has a much narrower measurement error histogram, with the majority of values falling within ±0.05m.

TABLE 6
Errors of SVR UWB measurements when
compared to GAITRite ground truth
measurements

| Metric | RMSE | MAE | MAPE |
|---|---|---|---|
| Step Width (m) | 0.034 | 0.029 | 4.35% |
| Step Length (m) | 0.037 | 0.031 | 4.70% |
| Stride Length (m) | 0.043 | 0.035 | 2.70% |

### Multilayer Perceptron

In a third example of a sensor fusion process, the UWB data and IMU data may be combined via a Multilayer Perceptron (MLP).

A regression MLP model was built using the Sequential API from Keras. The same 113 extracted features as the SVR model may be used, with the same preprocessing. 10-fold cross validation may also be used to select the best performing MLP model. Through experimentation it was found that for step length, width, and stride length, the best performing model was a single layer MLP with two nodes. The activation function was the Relu function.

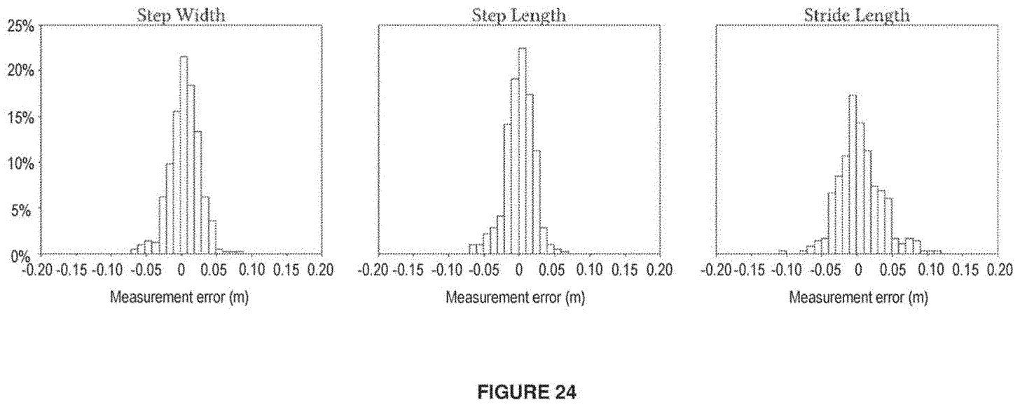

The hyper parameters for MLP were the number of layers, number of nodes, and the activation functions used. Again, 10-fold cross validation was used and the steps dataset was divided into ten partitions such that a similar percentage of the amount of steps from each participant was in each partition. To make sure this result was stable we trained 100 different MLPs, and the results can be seen in Table 7 and in FIG. 24. We report the Root Mean Square Error (RMSE), Mean Absolute Error (MAE), and Mean Absolute Percentage Error (MAPE) and their standard deviations.

Importantly, the MLP model achieves MAPE of 2.26% in estimating step width, 2.24% in estimating step length, and 2.49% in estimating stride length when comparing to the GAITRite. Note that compared to all previous models the MLP model has narrowest measurement error histograms, with the vast majority of measurements falling within ±0.03m.

TABLE 7
Errors of MLP UWB measurements when compared to
GAITRite ground truth measurements

| Metric | RMSE (SD) | MAE (SD) | MAPE (SD) |
|---|---|---|---|
| Step Width (m) | 0.020 (0.002) | 0.015 (0.001) | 2.26% (0.19%) |
| Step Length (m) | 0.020 (0.001) | 0.015 (0.001) | 2.24% (0.15%) |
| Stride Length (m) | 0.043 (0.011) | 0.033 (0.009) | 2.49% (0.69%) |

In view of the above, it can be seen that by using sensor fusion, it is possible to measure important gait metrics not previously measurable in traditional IMU-based sensors, such as step width. We compared three approaches to sensor fusion EKF, SVR, and MLP with MLP performing the best for spatial metrics. Indeed, these MLP derived metrics are close to the GAITRite reported accuracy of ±1.27 cm. Importantly, embodiments of the present invention are able to estimate step width which is predictive of fall risk and is thus of great clinical significance. The best methods for calculating the gait metrics detailed herein can be seen in Table 8.

Whilst the EKF method is not as accurate as the MLP and SVR methods, it is more interpretable. Specifically, EKF filtering can be used to estimate foot movement while in the air as the shape of the UWB signal is better preserved than by other filtering methods. Additionally, the EKF can be improved by the use of quaternions, and integrating the other accelerometer axes into the model. Another possible improvement is combining all four device pairs into one EKF model, with constraints based on the fixed positioning of the devices on the shoe. Further experimentation and simulation could allow a more correct model of the system noise Q.

TABLE 8
Best Performing UWB measurements when compared
to GAITRite ground truth measurements

| Metric | Method | RMSE | MAE | MAPE |
|---|---|---|---|---|
| Step Time (s) | Baseline | 0.016 | 0.012 | 2.14% |
| Stride Time (s) | Baseline | 0.016 | 0.011 | 1.01% |
| Step Width (s) | MLP | 0.020 | 0.015 | 2.26% |
| Step Length (m) | MLP | 0.020 | 0.015 | 2.24% |
| Stride Length (m) | MLP | 0.043 | 0.033 | 2.49% |
| Stride Velocity (m/s) | Baseline | 0.067 | 0.051 | 4.23% |
| Cadence (steps/min) | Baseline | 0.703 | 0.442 | 0.42% |

An important benefit of the above approach is that every single algorithm used herein could feasibly run on the embedded hardware available in the sensor units (such as sensor unit u₁ shown in FIG. 2). In both the baseline and EKF methods, the most computationally expensive step is the filtering approach native to each method. However, lightweight methods exist for both of these filtering approaches, namely Microsmooth for Savitzky-Golay and TinyEKF5 for EKF. The SVR models used herein have either a linear or sigmoid kernel and could be implemented on the sensor unit using Arduino-SVM6. Finally, the MLP model selected was very small with only two nodes and fewer than three hundred parameters, and therefore could be executed even on underpowered hardware.

This system 10 allows the user to walk in any direction during measurement, unlike the GAITRite which only allows for straight line walking. It requires no room to be set up unlike motion capture systems which require dedicated space for use. The system 10 is inexpensive compared to other devices and due to its form factor is very convenient to use. Embodiments of the present invention may have use in many applications, for example, in tracking motion in sports medicine, gait-based neurological diagnostics for conditions such as Parkinson's disease, fall risk and frailty assessment, and the monitoring of the elderly.

Embodiments of the sensor system 10 are capable of measuring important gait metrics not previously measurable in traditional IMU-based sensors. Step width variability in particular is predictive of fall risk and thus of great importance. Furthermore, the sensors of system 10 can measure foot positions on step placement. This could be used to detect step abnormality.

It will be appreciated that many further modifications and permutations of various aspects of the described embodiments are possible. Accordingly, the described aspects are intended to embrace all such alterations, modifications, and variations that fall within the spirit and scope of the appended claims.

Embodiments of the invention may, for example, comprise features in accordance with the following numbered statements:
1. A system for motion analysis of a subject, including:
   two or more sensor units that are attachable at respective attachment points of the subject to detect motion of the attachment points relative to each other, each sensor unit including a time-of-flight (TOF) ranging sensor in communication with at least one processor;
   wherein the at least one processor is configured to:
      cause the sensor units to execute a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
      determine, from at least the TOF distance data, one or more motion metrics.
2. A system according to Statement 1, wherein at least one of said processors is external to said sensor units.
3. A system according to Statement 1 or Statement 2, wherein the sensor units include:
   a first sensor unit and a second sensor unit for placement in spaced arrangement at a first spacing on a first foot of the subject; and
   a third sensor unit and a fourth sensor unit for placement in spaced arrangement at a second spacing on a second foot of the subject.
4. A system according to any one of Statements 1 to 3, wherein the two-way ranging protocol is a single-sided two-way ranging protocol that includes:
   a, or the, first sensor unit transmitting a first poll signal to each other sensor unit;
   one or more of the other sensor units transmitting a response signal, or response signals, to the first sensor unit, each said response signal including a difference between a time of receipt of the first poll signal at the respective other sensor unit and a time of transmission of the response signal.
5. A system according to Statement 3, wherein the two-way ranging protocol is a single-sided two-way ranging protocol that includes:
   the first sensor unit transmitting a first poll signal to the second, third and fourth sensor units;
   the second sensor unit transmitting a second poll signal to the third and fourth sensor units;
   the third sensor unit transmitting a first response signal to the first and second sensor units, the first response signal including a difference between a time of receipt of the first poll signal at the third sensor unit and a time of transmission of the first response signal, and a difference between a time of receipt of the second poll signal at the third sensor unit and the time of transmission of the first response signal; and
   the fourth sensor unit transmitting a second response signal to the first and second sensor units, the second response signal including a difference between a time of receipt of the first poll signal at the fourth sensor unit and a time of transmission of the second response signal, and a difference between a time of receipt of the second poll signal at the fourth sensor unit and a time of transmission of the second response signal.
6. A system according to Statement 4 or Statement 5, wherein the first poll signal includes a beacon time.
7. A system according to any one of Statements 4 to 6, wherein the one or more respective distances are determined only by the processor of the first sensor unit and, if applicable, the processor of the second sensor unit.
8. A system according to any one of Statements 1 to 7, wherein the TOF ranging sensors are RF ranging sensors.
9. A system according to Statement 8, wherein the RF ranging sensors are ultra wideband (UWB) sensors.
10. A system according to any one of Statements 1 to 9, wherein each sensor unit is configured to record a respective sensor temperature; and wherein the at least one processor is configured to adjust the respective distances using the respective sensor temperatures and a temperature calibration model.
11. A system according to any one of Statements 1 to 10, wherein at least one of the sensor units further includes an inertial sensor in communication with the at least one processor, each said inertial sensor being configured to measure inertial sensor data including at least accelerometer data and gyroscope data.
12. A system according to any one of Statements 1 to 11, wherein the at least one processor is configured to apply a smoothing filter to at least the TOF distance data.
13. A system according to Statement 12, wherein the filter is a Savitsky-Golay filter or an extended Kalman filter.
14. A system according to any one of Statements 11 to 13, wherein the at least one processor is configured to determine the one or more motion metrics by a sensor fusion process that combines the TOF distance data and the inertial sensor data.
15. A system according to Statement 14, wherein the sensor fusion process includes extracting a plurality of features from the TOF distance data and the inertial sensor data, and applying at least one machine learning model to the plurality of features to determine the one or more motion metrics.
16. A system according to Statement 15, wherein the at least one machine learning model includes a support vector regression model or a multilayer perceptron model.
17. A system according to any one of Statements 1 to 16, wherein the one or more motion metrics include one or more gait metrics.
18. A system according to any one of Statements 1 to 17, wherein the at least one processor is configured to determine one or more stationary points of a curve defined at least partly by the TOF distance data and/or the inertial sensor data, or a smoothed version thereof; and wherein at least one of said motion metrics is computed based on the one or more stationary points.
19. A method of motion analysis of a subject, including:
   attaching two or more sensor units at respective attachment points of the subject to detect motion of the attachment points relative to each other, each sensor unit including a time-of-flight (TOF) ranging sensor in communication with at least one processor;
   executing, by the at least one processor, a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
   determining, from at least the TOF distance data, one or more motion metrics.
20. A method according to Statement 19, including:
   attaching a first sensor unit and a second sensor unit at a first spacing from each other on a first foot of the subject; and
   attaching a third sensor unit and a fourth sensor unit at a second spacing from each other on a second foot of the subject.
21. A method according to Statement 19 or Statement 20, wherein the two-way ranging protocol is a single-sided two-way ranging protocol that includes:
   a, or the, first sensor unit transmitting a first poll signal to each other sensor unit;
   one or more of the other sensor units transmitting a response signal, or response signals, to the first sensor unit, each said response signal including a difference between a time of receipt of the first poll signal at the respective other sensor unit and a time of transmission of the response signal.
22. A method according to Statement 20, wherein the two-way ranging protocol is a single-sided two-way ranging protocol that includes:
   the first sensor unit transmitting a first poll signal to the second, third and fourth sensor units;
   the second sensor unit transmitting a second poll signal to the third and fourth sensor units;
   the third sensor unit transmitting a first response signal to the first and second sensor units, the first response signal including a difference between a time of receipt of the first poll signal at the third sensor unit and a time of transmission of the first response signal, and a difference between a time of receipt of the second poll signal at the third sensor unit and the time of transmission of the first response signal; and
   the fourth sensor unit transmitting a second response signal to the first and second sensor units, the second response signal including a difference between a time of receipt of the first poll signal at the fourth sensor unit and a time of transmission of the second response signal, and a difference between a time of receipt of the second poll signal at the fourth sensor unit and a time of transmission of the second response signal.
23. A method according to Statement 21 or Statement 22, wherein the first poll signal includes a beacon time.
24. A method according to any one of Statements 21 to 23, wherein the one or more respective distances are determined only by the processor of the first sensor unit and, if applicable, the processor of the second sensor unit.
25. A method according to any one of Statements 19 to 24, wherein the TOF ranging sensors are RF ranging sensors.
26. A method according to Statement 25, wherein the RF ranging sensors are ultra wideband (UWB) sensors.
27. A method according to any one of Statements 19 to 26, including recording, by each of said sensor units, a respective sensor temperature; and adjusting the respective distances using the respective sensor temperatures and a temperature calibration model.
28. A method according to any one of Statements 19 to 27, wherein at least one of the sensor units further includes an inertial sensor in communication with the at least one processor, and wherein the method includes measuring, by said inertial sensor, inertial sensor data including at least accelerometer data and gyroscope data.
29. A method according to any one of Statements 19 to 28, including applying a smoothing filter to at least the TOF distance data.
30. A method according to Statement 29, wherein the filter is a Savitsky-Golay filter or an extended Kalman filter.
31. A method according to any one of Statements 28 to 30, including determining the one or more motion metrics by a sensor fusion process that combines the TOF distance data and the inertial sensor data.
32. A method according to Statement 31, wherein the sensor fusion process includes extracting a plurality of features from the TOF distance data and the inertial sensor data, and applying at least one machine learning model to the plurality of features to determine the one or more motion metrics.
33. A method according to Statement 32, wherein the at least one machine learning model includes a support vector regression model or a multilayer perceptron model.
34. A method according to any one of Statements 19 to 33, wherein the one or more motion metrics include one or more gait metrics.
35. A method according to any one of Statements 19 to 34, including determining one or more stationary points of a curve defined at least partly by the TOF distance data and/or the inertial sensor data, or a smoothed version thereof; and computing at least one of said motion metrics based on the one or more stationary points.
36. At least one computer-readable medium storing machine-readable instructions that, when executed by at least one processor, cause the at least one processor to perform a method according to any one of Statements 19 to 35.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A system for motion analysis of a subject, including:
two or more sensor units that are attachable at respective attachment points of the subject to detect motion of the attachment points relative to each other, each sensor unit including a time-of-flight (TOF) ranging sensor in communication with at least one processor;
wherein the at least one processor is configured to:
cause the sensor units to execute a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
determine, from at least the TOF distance data, one or more motion metrics.

2. A system according to claim 1, wherein the sensor units include:
a first sensor unit and a second sensor unit for placement in spaced arrangement at a first spacing on a first foot of the subject; and
a third sensor unit and a fourth sensor unit for placement in spaced arrangement at a second spacing on a second foot of the subject.

3. A system according to claim 1, wherein the two-way ranging protocol is a single-sided two-way ranging protocol that includes:
a, or the, first sensor unit transmitting a first poll signal to each other sensor unit;
one or more of the other sensor units transmitting a response signal, or response signals, to the first sensor unit, each said response signal including a difference between a time of receipt of the first poll signal at the respective other sensor unit and a time of transmission of the response signal.

4. A system according to claim 2, wherein the two-way ranging protocol is a single-sided two-way ranging protocol that includes:
the first sensor unit transmitting a first poll signal to the second, third and fourth sensor units;
the second sensor unit transmitting a second poll signal to the third and fourth sensor units;
the third sensor unit transmitting a first response signal to the first and second sensor units, the first response signal including a difference between a time of receipt of the first poll signal at the third sensor unit and a time of transmission of the first response signal, and a difference between a time of receipt of the second poll signal at the third sensor unit and the time of transmission of the first response signal; and
the fourth sensor unit transmitting a second response signal to the first and second sensor units, the second response signal including a difference between a time of receipt of the first poll signal at the fourth sensor unit and a time of transmission of the second response signal, and a difference between a time of receipt of the second poll signal at the fourth sensor unit and a time of transmission of the second response signal.

5. A system according to claim 3, wherein the first poll signal includes a beacon time.

6. A system according to claim 3, wherein the one or more respective distances are determined only by the processor of the first sensor unit and, if applicable, the processor of the second sensor unit.

7. A system according to claim 1, wherein each sensor unit is configured to record a respective sensor temperature; and wherein the at least one processor is configured to adjust the respective distances using the respective sensor temperatures and a temperature calibration model.

8. A system according to claim 1, wherein at least one of the sensor units further includes an inertial sensor in communication with the at least one processor, each said inertial sensor being configured to measure inertial sensor data including at least accelerometer data and gyroscope data.

9. A system according to claim 8, wherein the at least one processor is configured to determine the one or more motion metrics by a sensor fusion process that combines the TOF distance data and the inertial sensor data.

10. A system according to claim 1, wherein the one or more motion metrics include one or more gait metrics.

11. A system according to claim 1, wherein the at least one processor is configured to determine one or more stationary points of a curve defined at least partly by the TOF distance data and/or the inertial sensor data, or a smoothed version thereof; and wherein at least one of said motion metrics is computed based on the one or more stationary points.

12. A method of motion analysis of a subject, including:
attaching two or more sensor units at respective attachment points of the subject to detect motion of the attachment points relative to each other, each sensor unit including a time-of-flight (TOF) ranging sensor in communication with at least one processor;
executing, by the at least one processor, a two-way ranging protocol at a succession of times, said two-way ranging protocol including transmission of one or more signals from, and reception of one or more signals at, said TOF ranging sensors, to determine TOF distance data indicative of one or more respective distances between the sensor units at respective times; and
determining, from at least the TOF distance data, one or more motion metrics.

13. A method according to claim 12, wherein the two-way ranging protocol is a single-sided two-way ranging protocol that includes:
a, or the, first sensor unit transmitting a first poll signal to each other sensor unit;
one or more of the other sensor units transmitting a response signal, or response signals, to the first sensor unit, each said response signal including a difference between a time of receipt of the first poll signal at the respective other sensor unit and a time of transmission of the response signal.

14. A method according to claim 12, comprising attaching a first sensor unit and a second sensor unit at a first spacing from each other on a first foot of the subject; and attaching a third sensor unit and a fourth sensor unit at a second spacing from each other on a second foot of the subject; wherein the two-way ranging protocol is a single-sided two-way ranging protocol that includes:
the first sensor unit transmitting a first poll signal to the second, third and fourth sensor units;
the second sensor unit transmitting a second poll signal to the third and fourth sensor units;
the third sensor unit transmitting a first response signal to the first and second sensor units, the first response signal including a difference between a time of receipt of the first poll signal at the third sensor unit and a time of transmission of the first response signal, and a difference between a time of receipt of the second poll signal at the third sensor unit and the time of transmission of the first response signal; and
the fourth sensor unit transmitting a second response signal to the first and second sensor units, the second response signal including a difference between a time of receipt of the first poll signal at the fourth sensor unit and a time of transmission of the second response signal, and a difference between a time of receipt of the second poll signal at the fourth sensor unit and a time of transmission of the second response signal.

15. A method according to claim 13, wherein the first poll signal includes a beacon time.

16. A method according to claim 12, including recording, by each of said sensor units, a respective sensor temperature; and adjusting the respective distances using the respective sensor temperatures and a temperature calibration model.

17. A method according to claim 12, wherein at least one of the sensor units further includes an inertial sensor in communication with the at least one processor, and wherein the method includes measuring, by said inertial sensor, inertial sensor data including at least accelerometer data and gyroscope data.

18. A method according to claim 17, including determining the one or more motion metrics by a sensor fusion process that combines the TOF distance data and the inertial sensor data.

19. A method according to claim 12, wherein the one or more motion metrics include one or more gait metrics.

20. A method according to claim 12, including determining one or more stationary points of a curve defined at least partly by the TOF distance data and/or the inertial sensor data, or a smoothed version thereof; and computing at least one of said motion metrics based on the one or more stationary points.
